(19) United States
(12) Patent Application Publication
(10) Pub. No.: US 2008/0107660 A1
(43) Pub. Date: May 8, 2008

(54) Binding Agents

(76) Inventor: Colin Self, Northumberland (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/590,840

(22) PCT Filed: Feb. 25, 2005
(86) PCT No.: PCT/GB05/00704

§ 371 Date: Aug. 17, 2007

(30) Foreign Application Priority Data
Feb. 25, 2004 (GB) 0404187.7

(51) Int. Cl. A61K 39/395; C07K 16/18; C07K 14/00; C07D 235/02; G01N 33/542; G01N 33/567; C12N 5/06; A61P 43/00; A61K 38/43; G01N 33/574; G01N 33/53; G01N 33/00; C12Q 1/68

(52) U.S. Cl. 424/172.1; 530/391.1; 530/367; 548/303.7; 435/7.9; 435/7.2; 435/7.23; 435/7.22; 435/7.24; 436/501; 435/7.92; 435/6; 424/94.1; 435/375

(57) ABSTRACT

The invention relates to binding agents which can be induced to aggregate in response to a specific stimulus. In preferred embodiments the binding agents comprise at least a binding member for a binding partner and the binding partner. At least one of the binding member and binding partner are reversibly masked such that a plurality of binding agents are not able to aggregate with one another until the masked moiety is unmasked. Preferred mechanisms to induce aggregation include irradiation and enzyme action. Applications of the invention include signal amplification in biochemical assays and provision of high local concentrations of therapeutic agents in vivo.

### FIELD OF THE INVENTION

The present invention relates to binding agents, and in particular to binding agents which can be induced to aggregate in response to a specific stimulus. Such reagents find use in both diagnostic and therapeutic applications, inter alia for signal amplification, as well as in microfabrication technology where aggregation of components is required.

### BACKGROUND TO THE INVENTION

Immunodiagnostic methods have proved to be of great use both within and outside of clinical areas. Food testing, environmental testing and forensic applications are but some of the applications. The robustness, precision and convenience of the methods have led to applications ranging from kits for home use to sophisticated laboratory auto-analysers. In particular, the development of immunometric technologies for large molecular weight analytes has been outstandingly successful.

Immunoassays frequently use antibodies to detect immobilised analyte. These antibodies are conventionally coupled to fluorescent label molecules, or (e.g. in an ELISA) to enzymes capable of producing a colour change by acting on a substrate, to provide a spectrophotometrically detectable readout. Such enzymes include alkaline phosphatase and horseradish peroxidase). Thus the signal produced depends on the number of antibody molecules bound to the immobilised analyte. However typically the signal produced is relatively low per antibody molecule bound. This can lead to a lack of sensitivity, particularly when detecting low concentrations of analyte. Attempts have been made to increase the signal produced by such enzyme-based assays, e.g. by coupling chains of enzyme molecules to detection reagents, or using multiple detection layers. With pre-formed chains of molecules, diffusion and binding to the target may be hindered by size and other steric effects. Therefore it may be advantageous to use smaller molecules which assemble at or close to the required site. Use of multiple detection layers, added sequentially, is a well used method of increasing the number of detector molecules brought to a site. While it is an effective method of boosting detectable signal it increases complexity, is time consuming, relatively cumbersome with the added disadvantage of introducing the possibility of error. Methods of increasing the amount of detector molecule by secondary precipitation of secondary label have been developed such as Catalysed Reporter Deposition (the Tyramide signal amplification method) in which a first enzyme label gives rise to an insoluble moiety which after precipitation around the site can then be detected by the subsequent addition of a secondary antibody against it. While these methods are capable of increasing signal they also increase complexity with respect to the final user operation concerned, for example, when sequential additions of the various reagents are required. Thus there is a need for further methods of amplifying signals in such assays which are both effective and user-friendly.

Antibodies have also been proposed for targeting therapeutic agents to desired disease sites. This approach is, for example, used for targeting an enzyme capable of converting a prodrug into an active drug to a specific site. An antibody directed against a molecule expressed specifically at that site is administered to the subject. The enzyme may be coupled to that antibody, or to a secondary antibody directed against the first and administered subsequently. This type of therapy is sometimes known as ADEPT (antibody directed enzyme prodrug therapy). Methods of increasing the amount of active enzyme at the desired site, and of increasing the temporal and spatial specificity of activation of the prodrug, are highly desirable. Agents capable of targeted aggregation may find many other possible uses in therapeutic areas where a therapeutic agent is to be targeted to a given site or activated at a selected time.

### SUMMARY OF THE INVENTION

The present invention provides reagents which can be triggered to self-assemble or aggregate by administration of a suitable stimulus. The stimulus can be administered (or simply encountered by the reagents) in a temporally and spatially specific manner, leading to fine control of aggregation. Reagents of the invention can be used in a wide variety of diagnostic, therapeutic, and other applications.

In a first aspect the present invention provides a binding agent comprising two binding moieties, the binding moieties comprising

(i) a binding member, capable of binding to a binding partner, and
(ii) the binding partner,
one of said binding moieties being reversibly masked, whereby two such binding agents do not bind one another. The binding agents become capable of binding one another once the reversibly masked binding moiety is unmasked, and so form a complex or aggregate.

Also provided is a method of causing aggregation of a plurality of binding agents, comprising providing a composition comprising a plurality of binding agents, each comprising two binding moieties, the binding moieties comprising

(i) a binding member for a binding partner, and
(ii) the binding partner,
one of said binding moieties being reversibly masked, whereby said plurality of binding agents do not bind one another,
and unmasking said binding moiety, whereby said plurality of binding agents become capable of binding to one another.

The two binding moieties are preferably covalently linked, and so form part of the same molecule. However the binding agent may also be a molecular complex held together by non-covalent interactions, with the two binding moieties being located on different components of the complex. Reference herein to a binding agent molecule should be construed accordingly.

Thus in this aspect of the invention the binding agent comprises two binding moieties which in isolation are capable of binding to one another, and hence are capable of inducing aggregation of a plurality of identical such binding agents. One (or more) of these moieties is masked so that such aggregation cannot take place until that moiety is unmasked. These complementary binding moieties are referred to herein as a binding member and a binding partner.

However, the terms “binding member” and “binding partner” should not be taken to imply any particular structural or functional relationship other than the capacity of each to bind the other. Furthermore, it will be understood that a moiety referred to as a binding member in one context may equally be referred to as a binding partner in another context.

Typically the binding member and binding partner are members of a specific binding pair, as described in more detail below. Suitable binding pairs include, but are not restricted to, antibody and cognate antigen (which may be a whole antigen or a fragment thereof as long as it comprises the epitope to which the antibody binds), receptor and ligand, avidin/streptavidin and biotin, nucleic acids, carbohydrates and lectins, etc.

Preferably, the moieties of a single binding agent cannot interact with one another after the binding moiety is unmasked. Such intra-molecular interactions are generally undesirable because they will tend to reduce the potential for inter-molecular aggregation. Typically, then, the binding moieties will be spaced apart such that they are sterically incapable of interacting with one another, but are capable of interacting with their respective complementary binding moieties on other binding agents.

The degree to which cross-linking occurs within the aggregate of binding agents will depend on the number of each type of binding moiety present on each binding agent. Each binding agent may therefore comprise a plurality of one or both types of binding moiety; for example, they may independently comprise two, three, four, five or more copies of one or both binding moieties.

Unmasking the reversibly masked binding moiety may be regarded as activation of that moiety, or activation of the binding agent in general.

The appropriate binding moiety may be masked by a masking element located at or adjacent that binding moiety. The masking element may prevent binding to the cognate binding moiety via steric interference, electrostatic repulsion or any other suitable mechanism.

A detachable masking element may be provided; for example the masking element may be coupled to the binding agent via a selectively cleavable group or bond.

The masking element may be cleavable from the binding agent by a variety of techniques, including, but not limited to irradiation, oxidation, reduction, pH change, or enzymatic cleavage. The cleavage method may be selected appropriately depend upon the application for which the binding agent is to be used.

It may be desirable to design the binding agent such that the binding moiety is unmasked by action of a particular enzyme. For example, it may be desirable that the binding moiety is unmasked by an enzyme secreted by a particular tumour cell so that the binding (and/or effector) properties of the binding agent are activated only in proximity to that cell type. Suitable enzymes include proteases, such as metalloproteases, which are secreted by a variety of tumour cells, especially metastatic tumour cells.

Use of enzymatic cleavage as an activation mechanism may enable a chain reaction of binding agent activation to take place, wherein one activated binding agent is capable of activating a further binding agent, and so on. Particularly useful are binding agents comprising an inactive enzyme (e.g. a proenzyme or zymogen), wherein the active form of the enzyme is capable of converting its own inactive form to its active form, e.g. by cleaving the propetide from a proenzyme. Typically the binding agent will comprise a binding moiety (e.g. an antibody) which binds preferentially to the active form of the enzyme, and preferably does not bind, or substantially does not bind, to the inactive form. In such cases, the mask may be seen as the element causing the inactivity of the enzyme, e.g. the propeptide of a proenzyme.

An example of a suitable proenzyme is chymotrypsinogen, which is activated to chymotrypsin by cleavage of its propeptide. Chymotrypsin is itself capable of converting chymotrypsinogen to chymotrypsin.

Alternatively, the masked binding moiety may be unmasked by a conformational change in the binding agent. This may create a new conformation in the binding agent which can be bound by the binding member, or may expose a part of the binding agent previously masked or screened (e.g. sterically). Thus the binding moiety may be unmasked by a conformational change in the binding agent, whereby said binding member or said binding partner is exposed. Conformational change may take place along with, or as a result of, another method of activation. For example, where a proenzyme is used as a binding moiety as described above, the complementary binding moiety may recognise a particular conformation which is generated only on cleavage of the propeptide. An antibody capable of binding specifically to chymotrypsin but not to chymotrypsinogen may thus be used in combination with chymotrypsinogen.

Thus the mask may be removed or reversed by a conformational change in the binding agent, whereby said binding moiety is exposed.

Irradiation is a further potential activation mechanism, which is particularly preferred, especially in therapeutic applications, because of the tight spatial and temporal control over activation which it provides. It is possible to administer a binding agent to a subject systemically or locally, and activate the binding agent very specifically e.g. by means of a laser. This may reduce undesirable activation elsewhere in the body. Thus one or both of the binding moieties may be masked by a photocleavable moiety; i.e. a moiety linked to the rest of the binding agent via a photocleavable bond.

The binding agent will typically comprise an effector member. The nature and identity of the effector will depend upon the intended use of the binding agent. The binding agents may be used in any application in which it is desirable to cause a localised increase in concentration of a particular effector, whether in vivo or in vitro, including laboratory assay systems and cell culture.

The binding agents described herein may be used to provide or amplify a signal, in an assay for determining the presence or concentration of an analyte at a particular site or in a sample. Such assays include biological, biochemical, or immunological assays performed in vitro, as well as diagnostic tests practised on an individual in vivo.

In such embodiments, the effector may comprise a signal generating means. Examples include label moieties, such as radiolabels or fluorescent labels. Alternatively the signal generating means may comprise an enzyme capable of acting on a substrate to produce a detectable readout. This may include acting on a substrate to produce a spectrophotometrically detectable change such as a colour change. Such enzymes include alkaline phosphatase and horseradish peroxidase. Other possibilities will be apparent to the skilled person.

In other embodiments the effector member may have a binding functionality. For example it may be capable of binding to a target cell type. In particular embodiments it may be capable of inducing aggregation or activation of one or more particular cell types at the site of binding agent aggregation. Thus the effector member may be capable of binding to the surface of a cell, and optionally activating that cell type. Thus the effector member may bind to, and optionally be an agonist of, a cell surface molecule. To illustrate, a molecule capable of binding to a molecule expressed on the surface of platelets such as CD41 (e.g. an anti-CD41 antibody) could be used to induce localised aggregation of platelets and so promote blood clotting. Where agonist activity is required, the effector may be a receptor, co-receptor, ligand or another type of agonist such as an antibody. The cell may be a cell of the immune system, such as a T cell or natural killer (NK) cell. For example, the effector may be an anti-CD3 antibody capable of activating T cells at the site of binding agent aggregation. Such effectors may be useful therapeutically, e.g. for activation of an immune response. This may be useful for treatment of cancers or infections by intracellular parasites, such as viruses, malaria, etc.

Effectors capable of binding to the surface of a target cell may also be used to localise the binding agents to that particular cell type, e.g. to target a therapeutically useful effector to that cell type. Binding agents such as antibodies which are capable of binding to cell-specific antigens are particularly useful here. Examples include binding agents for tumour specific antigens, and parasite antigens, such as viral antigens.

Other effectors which may be therapeutically useful include drug and prodrug molecules, as well as enzymes for converting prodrugs to an active form. Such enzymes include phosphatases, carboxypeptidase, beta-glucosidase, beta-lactamases, amidase, cytosine deaminase and nitroreductase. These may also be useful in the treatment of infections or cancers. Binding agent aggregation may be induced at the site of a tumour or infection, e.g. by irradiation, allowing localised concentration of the relevant effector.

The effector moiety may also be reversibly masked or inhibited to prevent it exerting its function until activated. The effector moiety may be masked by the same means as the binding member/partner, or by a different means. Thus the same or different stimuli may be needed to activate aggregation and effector functions.

The invention further provides methods of making binding agents as described herein. Preferred methods comprise the steps of:

(i) providing a first component comprising a binding member, capable of binding to a binding partner; and
(ii) providing a second component, comprising said binding partner;
wherein one of said binding member and binding partner is reversibly masked such that said binding member or partner is prevented from binding to the other; and
iii) contacting said first component with said second component such that they become associated with one another.

The method may comprise the further step of reversibly masking the relevant binding member or partner.

The first and second components may be associated by covalent bonds such that they form a single molecule. For example, conventional conjugation reagents may be used to form a covalent conjugate between the two components. Alternatively the components may be associated with one another non-covalently, e.g. to form a complex held together by non-covalent interactions, such as antibody-antigen or avidin-biotin interactions.

Each of said first and second components may comprise a plurality of said binding members or partners, only a fraction of which are masked. Thus they may be joined together via interaction between unmasked binding members and partners, as long as the resulting complex or molecule still comprises at least one free binding partner and at least one free binding member, one of which is reversibly masked.

For example, a single component complex may be generated by partially masking an avidin molecule, so that some, but not all, of its four biotin binding sites are masked and cannot bind biotin. The partially masked biotin is then contacted with a biotinylated effector molecule (e.g. alkaline phosphatase for use in amplifying a signal in an immunoassay). This leads to the formation of complexes which will themselves aggregate when the remaining avidin binding sites are unmasked.

The invention also extends to systems having two or more binding agent components. Where two or more components are used, any one binding agent need not carry both a binding member and its respective binding partner. Instead, each binding agent should carry at least two binding moieties, each being the binding partner for a binding member present on another component of the system (another binding agent). As implied above and elsewhere, the terms “binding member” and “binding partner” here can be regarded as interchangeable. Thus a “binding member” on a first component could equally be referred to as the “binding partner” of a complementary “binding member” on another component of the system.

Thus in a further aspect the present invention provides a composition comprising at least two populations of binding agents, each population having at least two binding members for respective binding partners, each of said binding partners being present on another of said populations of binding agents, wherein a binding member of at least one of said populations is reversibly masked, whereby said one population does not bind the population carrying the respective binding partner.

Also provided is a method of causing aggregation of a plurality of binding agents, comprising

obtaining a composition comprising at least two populations of binding agents, each population having at least two binding members for respective binding partners, each of said binding partners being present on another of said populations of binding agents, wherein a binding member of at least one of said populations is reversibly masked, whereby said one population does not bind the population carrying the respective binding partner,
and unmasking said binding member, whereby said one population becomes capable of binding the population carrying the respective binding partner.

Also provided is a kit comprising at least two populations of binding agents, each population having at least two binding members for respective binding partners, each of said binding partners being present on another of said populations of binding agents, wherein a binding member of at least one of said populations is reversibly masked, whereby said one population does not bind the population carrying the respective binding partner.

Each population comprises a plurality of a particular species of binding agent.

The binding members and partners of each binding agent may be the same or different.

Each population of binding agents may have one or more of its binding moieties masked. Each is preferably unmasked by the same mechanism.

As with the single-species binding agents described above, the degree to which cross-linking occurs within the aggregate of binding agents will depend on the number of each type of binding moiety present on each binding agent. Each binding agent may therefore comprise a plurality of one or both types of binding member or partner; for example, they may independently comprise two, three, four, five or more copies of one or both binding members or partners.

Preferred methods, kits and compositions comprise only two species of binding agent, in which each binding agent has (at least) two binding members/binding partners capable of binding to their complementary partner/member on the other binding agent. Thus in a preferred embodiment, each of the binding partners is present on the same other population of binding agents. Examples of two-component systems include

(i) an antibody having two antigen binding sites, in combination with an antigen molecule having at least two epitopes recognised by the antibody. Either the antigen binding sites or the cognate epitopes, or both, may be reversibly masked; (ii) an avidin molecule (having four biotin binding sites), in combination with a molecule conjugated to two or more biotin moieties. Typically, the avidin binding sites are reversibly masked to prevent interaction with biotin before activation. This may be regarded as a two-component version of the single-component binding agent described above which comprises a complex of partially masked avidin with a multiply biotinylated effector molecule.

The invention also encompasses methods, kits and compositions comprising three or more species of binding agent, in which at least one species is reversibly masked to prevent binding to at least one of the other species.

The multiple component systems, compositions, kits etc., may have masking and activation mechanisms, effector members etc. as described for the single component systems.

Systems, compositions and kits having two or more binding agents which associate together preferably contain substantially stoichiometrically equal amounts of each member of a particular binding pair. If one member is present in large excess, then the desired cross-linking and aggregation of binding agents is unlikely to occur. Instead, activation will lead to the formation of a large number of very small complexes, in which the binding agents carrying the binding member present in excess will (at most) be able to bind only one or a few of the binding agents carrying the respective binding partner. Efficient cross-linking requires that each binding agent associates with at least two other binding agents.

The methods of the invention may be applied in vitro or in vivo. Aggregation of binding agents may take place in solution or on a surface, e.g. a cell surface or the surface of a solid support such as a microtitre plate.

Where aggregation takes place on a surface, a binding member or binding partner may be present on that surface to enable a first binding agent to become associated with that surface. The binding agents may therefore aggregate at a specific focus, e.g. on a particular molecule present on or bound to that surface. Thus the methods of the invention may comprise the step of providing a focus for aggregation of the binding agents.

Typically the focus is a molecule comprising a moiety capable of binding to one of the binding agents, so that when activated (unmasked), the binding agents will aggregate at the focus.

This binding may be effected via one of the binding moieties present on the binding agents, i.e. a binding member or binding partner, or via an effector member of the binding agent, or any other part of the binding agent molecule. In preferred embodiments the focus for aggregation is a molecule which itself comprises at least one of the binding moieties present on the binding agents, i.e. a binding member or binding partner.

Additionally or alternatively, the method may comprise the step of providing a source of an agent capable of unmasking the masked binding moiety of the binding agent. This is particularly applicable where aggregation is not required to take place on a particular surface, but simply in the vicinity of a particular site, e.g. in free solution.

For example, the present invention provides means for causing targeted aggregation of binding agents carrying effector members having therapeutic properties, such as stimulation of the immune system, activation of prodrugs, etc. It may be desirable for such binding agents to aggregate in the vicinity of a disease site (e.g. a tumour, a parasitised cell, etc.), in order to increase activation of the immune system or a prodrug at that site. The binding agent may be designed such that its aggregation is triggered by an agent produced by or at the disease site, such as a viral enzyme produced by an infected cell, or a metalloprotease produced by a tumour cell. Thus the disease site itself may be regarded as the source of the activating agent.

The present invention further provides binding agents and compositions as described herein for use in a method of medical treatment, for example in the elimination or reduction of parasitised or tumour cells. Also provided is the use of binding agents and compositions as described herein in the manufacture of a medicament, e.g. for the treatment of cancer, or an infection by an intracellular parasite such as a virus or malaria.

As already described, a particularly preferred aspect of the invention relates to the use of the binding agents described above in assays to detect analytes in samples, in which the binding agents may serve as signal amplification reagents.

Thus the invention provides a method of determining an analyte in a sample, the method comprising

(a) contacting the sample with a plurality of binding agents each comprising two binding moieties, the binding moieties comprising
(i) a binding member, capable of binding to a binding partner, and
(ii) the binding partner,
one of said binding moieties being reversibly masked, whereby said binding agents do not bind one another;
   (b) contacting the sample with a detecting agent capable of binding to the analyte if present, wherein the detecting agent comprises one of said binding moieties; and
   (c) unmasking said reversibly masked binding moiety, whereby said binding agents form an aggregate.

Thus, if analyte is present in the sample, some of the binding agents will form a complex or aggregate associated with (e.g. bound to) the detecting agent and thus to the analyte. The detecting agent thus provides the “focus” for aggregation described above.

In certain embodiments, the analyte may comprise one of the binding moieties carried by the binding agents. In such cases the analyte itself provides the focus for aggregation. It is therefore not necessary to use a separate detecting agent. Thus, a binding agent which associates directly with (binds directly to) the analyte itself can be considered to be the detecting agent. Thus the detecting agent may be one of the binding agents.

The method typically comprises the step of detecting the presence of aggregated binding agents. This may be by direct or indirect means. The binding agents may carry signal generating means (e.g. a label moiety) and be detected as described elsewhere in the specification. Alternatively, the formation of an aggregate of binding agents may be determined directly by various methods including light scattering or surface plasmon resonance.

The method may involve immobilisation of the analyte on a solid phase, which will normally take place prior to contact with the detecting agent. For example, the sample may be coated directly onto a solid phase surface. Alternatively an immobilising agent specific for the analyte may itself be immobilised on the solid phase, providing binding sites to which the analyte may bind. The immobilising agent binds to a site on the analyte which is different to that recognised by the detecting agent, such that both the immobilising agent and detecting agent can bind simultaneously to the analyte. Typically the immobilising agent is an antibody specific for the analyte, although it may be a receptor or other suitable molecule.

In the assay methods described above, the aggregate of binding agents is bound or otherwise linked (e.g. via the detecting agent) to the analyte to be detected. This is desirable so that the bound aggregate is retained through washing steps intended to remove excess unreacted or unbound components at various stages of the assay. The method may include such washing steps as desired, e.g. between addition of detecting agent and addition of binding agents, and/or between activation of binding agents and detection of aggregate. It will be appreciated that the various components of the assay may be added simultaneously or sequentially as desired.

Preferred assay formats include immunochemical methods in which the detecting agent is an antibody specific for the analyte. these include methods such as ELISA (Enzyme Linked ImmunoSorbent Assay) and Western blots, but also assays such as immunohistochemistry and immunofluorescence where the analyte to be determined is found in a population of immobilised cells or in a tissue section.

Likewise, the methods described above can be applied to nucleic acid detection techniques where the analyte is a nucleic acid molecule (e.g. single or double stranded DNA or RNA, including genomic DNA, mRNA or cDNA) and the detecting agent is a nucleic acid molecule capable of hybridising under suitable stringency conditions (e.g. high, medium or low stringency conditions) with the analyte nucleic acid. Such techniques include Southern and Northern blots in which the analyte nucleic acid is immobilised on a membrane, as well as in situ hybridisation.

For example, a probe (detecting agent) for in situ hybridisation may be labelled with fluorescein. The signal from the fluorescein may be amplified by use of a binding agent consisting of a masked anti-fluorescein antibody conjugated to fluorescein. Unmasking of the antibody will result in aggregation of the fluorescein-containing binding agent on the probe, providing significantly greater signal than would be achieved via the probe alone.

It will be apparent that the sample will often be a solution (e.g. an aqueous solution, cell extract, or the like) suspected of containing the analyte. However, the methods of the invention are perfectly applicable to use with other types of sample such as cell preparations, tissue sections, etc. The analyte may be any suitable analyte, including, but not limited to proteins, polypeptides, peptide, carbohydrates, nucleic acids, organic or inorganic polymers, small organic or inorganic molecules, and larger entities including macromolecular complexes, viruses and eukaryotic or prokaryotic cells.

The methods and compositions of the invention can therefore be used to detect the presence of analytes (such as infectious agents like viruses or bacteria) in samples. Such assays may be performed in any suitable format, including homogeneous liquid assay formats. A simple precipitation or agglutination assay can be used, in which binding agents having an effector moiety capable of binding to the analyte are employed. The sample is contacted with the binding agent, the binding agent is activated (unmasked), e.g. by irradiation, and the rate and extent of precipitation observed, e.g. by light scattering or the like. The rate and extent of precipitation will depend on the amount of analyte in the sample.

In an alternative arrangement, a competitive assay format may be used. Typically, a sample containing an unknown amount of analyte is contacted with a known amount of a competitor. The analyte and competitor then compete for a fixed number of binding sites, and the concentration of analyte in the sample is calculated by determining the fraction of binding sites occupied by competitor.

Thus the invention provides a method of determining an analyte in a sample, the method comprising

(a) contacting the sample with
(i) a competitor, and
(ii) a plurality of binding sites, wherein each binding site is capable of binding to the analyte if present and to the competitor, but not to both simultaneously;
   (b) contacting the sample with a plurality of binding agents each comprising two binding moieties, the binding moieties comprising
(i) a binding member, capable of binding to a binding partner, and
(ii) the binding partner,
one of said binding moieties being reversibly masked, whereby said binding agents do not bind one another; and
   (c) contacting the sample with a detecting agent capable of binding to the competitor if present, wherein the detecting agent comprises one of said binding moieties; and
   (d) unmasking said reversibly masked binding moiety, whereby said binding agents form an aggregate.

The binding sites referred to in part (a)(ii) are typically part of immobilising agents (e.g. receptors or antibodies) immobilised on a solid phase as described above in relation to non-competitive assay systems.

It will be apparent to the skilled reader that, in the assay methods described above, binding agent systems having two or more binding agent components (as described above) may be used in place of the single binding agent species.

Thus the invention further provides a method of determining an analyte in a sample, the method comprising

(a) contacting the sample with at least two populations of binding agents, each population having at least two binding members for respective binding partners, each of said binding partners being present on another of said populations of binding agents, wherein a binding member of at least one of said populations is reversibly masked, whereby said one population does not bind the population carrying the respective binding partner, and
(b) contacting the sample with a detecting agent capable of binding to the analyte if present, wherein the detecting agent comprises one of said binding members or binding partners,
(c) unmasking said reversibly masked binding member, whereby said binding agents form an aggregate.

Also provided is a method of determining an analyte in a sample, the method comprising

(a) contacting the sample with
(i) a competitor, and
(ii) a plurality of binding sites, wherein each binding site is capable of binding to the analyte if present and to the competitor, but not to both simultaneously;
   (b) contacting the sample with at least two populations of binding agents, each population having at least two binding members for respective binding partners, each of said binding partners being present on another of said populations of binding agents, wherein a binding member of at least one of said populations is reversibly masked, whereby said one population does not bind the population carrying the respective binding partner;
   (c) contacting the sample with a detecting agent capable of binding to the competitor if present, wherein the detecting agent comprises one of said binding members or binding partners; and
   (d) unmasking said reversibly masked binding moiety, whereby said binding agents form an aggregate.

Preferred aspects of these methods are as described above and elsewhere in this specification.

A further particularly preferred aspect of the invention relates to the use of the binding agents described above in therapy, to provide a high local concentration of a particular therapeutic agent at a physiological site.

Thus the present invention provides a method of causing aggregation of a therapeutic agent at a physiological site, the method comprising administering a plurality of binding agents to an individual,

each of said binding agents comprising two binding moieties, the binding moieties comprising
(i) a binding member, capable of binding to a binding partner, and
(ii) the binding partner,
   one of said binding moieties being reversibly masked, whereby said binding agents do not bind one another, each of said binding agents further comprising said therapeutic agent,
   and wherein the method further comprises the step of inducing aggregation of said binding agents by unmasking said reversibly masked binding moiety such that said binding agents bind one another.

The physiological site may be a disease site, e.g. a site of a tumour or parasitic infection. Alternatively it may be a site where, for example, blood clotting is desirable, such as the site of a wound.

It may be desirable for the aggregate to be physically linked to the site (e.g. to an affected cell), although this is not always necessary.

Thus the method may comprise providing a focus on which the binding agents can aggregate. This may be achieved via a targeting agent. The targeting agent should be capable of binding to a molecule which is expressed (preferably specifically) at the site, and so may comprise an antibody (or fragment thereof) specific for a suitable molecule. Thus the targeting agent may be capable of binding to a tumour specific antigen or a parasite antigen (e.g. a viral antigen). Furthermore it should be capable of binding to one of the binding agents, and may therefore comprise one of the binding moieties carried by the binding agents.

The method may further comprise the step of administering the targeting agent to the individual. Additionally or alternatively, one of the binding moieties of the binding agent may be capable of binding to a molecule expressed at the site.

Unmasking of the binding moiety may be achieved by any suitable means. Preferred examples include illumination (where the binding moiety is masked by a photocleavable moiety) and enzyme action.

The enzyme involved in activation may be provided exogenously to the site (e.g. via a targeting agent of the type described above which is capable of binding to a molecule expressed at the site) or could be produced at the site. For example it could be an enzyme (e.g. a protease) expressed by a cancer cell, or a parasite enzyme (such as a viral enzyme) expressed by an infected cell. Thus it will be understood that the step of unmasking the binding moiety does not necessarily require a specific action on behalf of those administering the overall treatment. Unmasking (and hence aggregation) may simply occur when the binding agents encounter a site at which a suitable enzyme is expressed.

The therapeutic agents useful in these methods of the invention are described in more detail elsewhere in the specification. They include molecules capable of binding to molecules expressed on cell surfaces (e.g. to the surface of platelets), molecules capable of activating cells of the immune system (e.g. anti-CD3 antibodies), drug or prodrug molecules, and enzymes (e.g. an enzyme capable of converting a prodrug to an active form). In the latter case, the method may further comprise the step of administering the prodrug to the individual.

The therapeutic agent may also be reversibly masked so that it becomes active only when aggregation takes place, i.e. it only becomes active at the site.

It will be understood that in vitro methods corresponding to the therapeutic methods described above also fall within the scope of the invention.

Thus the invention also provides a method of causing aggregation of a therapeutic agent in a culture of cells, the method comprising contacting a culture of cells with a plurality of binding agents,

each of said binding agents comprising two binding moieties, the binding moieties comprising
(i) a binding member, capable of binding to a binding partner, and
(ii) the binding partner,
   one of said binding moieties being reversibly masked, whereby said binding agents do not bind one another,
   each of said binding agents further comprising said therapeutic agent,
   and wherein the method further comprises the step of inducing aggregation of said binding agents by unmasking said reversibly masked binding moiety such that said binding agents bind one another.

As set out in relation to therapeutic aspects of the invention, targeting agents may be employed if required. Other features of the therapeutic methods may also be adapted for use in such cell culture methods.

The cells may, for example, be cancer cells, parasitically-infected cells, cells of the immune system (such as T cells), platelets, etc.

It will be apparent to the skilled reader that, in the cell culture and therapeutic methods described above, binding agent systems having two or more binding agent components (as described above) may be used in place of the single binding agent species.

### DETAILED DESCRIPTION OF THE INVENTION

The invention provides materials and methods for producing aggregates of binding agents in a temporally and/or spatially specific manner by a specific stimulus resulting in the removal of a mask which prevents interaction between the binding agents. These materials and methods can be applied in numerous diagnostic and therapeutic settings in which fine control of aggregation is desirable.

### Specific Binding Pairs

The binding moieties described herein, i.e. a binding member and its complementary binding partner, preferably constitute a specific binding pair.

The term “specific binding pair” is used to describe a pair of molecules comprising a specific binding member (sbm) and a binding partner (bp) therefor which have particular specificity for each other and which in normal conditions bind to each other in preference to binding to other molecules. Examples of specific binding pairs are antibodies and their cognate epitopes/antigens, ligands (such as hormones, etc.) and receptors, avidin/streptavidin and biotin, lectins and carbohydrates, and complementary nucleotide sequences.

Enzymes may bind specifically to their substrate or to modulators such as inhibitors and activators, either competitive or allosteric, which may exert their physiological effects by binding to the active site or elsewhere on the enzyme molecule. Thus enzymes may be used as binding members in the present invention, although when the binding partner is a substrate for the enzyme, the enzyme may be inactivated so that it does not structurally alter or modify the binding partner. This may be achieved by mutagenesis (e.g. of a catalytically important residue at the active site), by removal of a cofactor, etc. Alternatively the enzyme may be unable to act on the substrate because of the absence in the assay medium of another required molecule such as a cosubstrate.

Molecular imprints may also be used as binding members. These may be made by forming a plastic polymer around a target analyte, extracting the analyte from the formed polymer, and then grinding the polymer to a fine powder, as described in Nonbiological Alternatives to Antibodies in Immunoassays; Principles and Practice of Immunoassay (second edition) Chapter 7 pp 139-153 Ed C P Price & D J Newman (1997).

Aptamers are DNA or RNA molecules, selected from libraries on the basis of their ability to bind other molecules. Aptamers have been selected which can bind to other nucleic acids, proteins, small organic compounds, and even entire organisms, and so may also be used in the present invention.

The skilled person will be able to think of many other examples and they do not need to be listed here.

The term “specific binding pair” is also applicable where either or both of the specific binding member and binding partner comprise just the binding part of a larger molecule. Thus in the context of antibodies, a specific binding member may comprise just a domain of an antibody (antibody binding domain) which is able to bind to either an epitope of an antigen or a short sequence which although unique to or characteristic of an antigen, is unable to stimulate an antibody response except when conjugated to a carrier protein.

It has been shown that fragments of a whole antibody can perform the function of binding antigens. The term “antibody” is therefore used herein to encompass any molecule comprising the binding fragment of an antibody. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding member (Bird et al, Science, 242, 423-426, 1988; Huston et al, PNAS USA, 85, 5879-5883, 1988).

Bispecific antibodies may also be used in the present invention. These include bispecific single chain Fv dimers (PCT/US92/09965) and “diabodies”, i.e. multivalent or multispecific fragments constructed by gene fusion (WO94/13804; P. Holliger et al., Proc. Natl. Acad. Sci. USA 90 6444-6448, 1993).

Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding member: antigen binding members are formed by the association of the first domain of one polypeptide within the multimer with the second domain of another polypeptide within the multimer (WO94/13804).

An example of a bispecific antibody which can be used as a binding agent according to the present invention is one having two antibody fragments in which a first antigen binding site is directed against a particular antigen, while a second antigen binding site is an anti-idiotypic site specific for the first antigen binding site. Such a bispecific antibody will aggregate when neither of the antigen binding sites is masked, and will also bind to the antigen for which the first fragment is specific. Alternatively the second binding site could be specific for an epitope on the first antibody fragment outside the antigen binding site, e.g. in the constant region of the first antibody fragment. The first antigen binding site may serve as the effector portion of the molecule, or a further effector portion may be present.

By way of illustration, the examples describe a T cell activation reagent which may be regarded as a bispecific antibody. It is composed of a murine anti-CD3 IgG antibody, coupled to an antibody directed against mouse IgG. Thus, in terms of the present invention, the bispecific antibody is a binding agent whose two binding moieties are the binding site of the anti-mouse IgG antibody, and the epitope on the anti-CD3 antibody for which it is specific. The anti-CD3 antibody binding site can be regarded as an effector moiety. The two binding moieties may be reversibly masked by coating one or both of the antibody components with a photocleavable moiety (e.g. NPE) before the two antibodies are linked together. The reagent can be made to aggregate by suitable illumination. If the anti-CD3 antibody is coated with the photocleavable reagent, then the anti-CD3 effector moiety will also be unable to interact with CD3 expressed on T cells until irradiation has taken place. Thus irradiation causes a local aggregation of the bispecific antibody and also unmasks the anti-CD3 effector moiety. The effect is to providing a complex of anti-CD3 antibody which is able to cross-link CD3 molecules expressed on nearby T cells and so stimulate T cell activation in that area. This may be useful to stimulate T cell activity in the vicinity of a tumour, for example.

A similar construct which comprises an anti-CD41 antibody instead of an anti-CD3 antibody may be used to promote blood clotting. CD41 is expressed on the surface of platelets. Activation of this construct in the bloodstream will therefore result in a local aggregation of platelets via the aggregated anti-CD41 binding sites. This may be useful at the site of a wound, or in order to restrict the supply of blood to a tumour.

### Masking and Activation Mechanisms

The binding agent(s) must be prevented from interacting with one another before the activating stimulus is delivered. Thus binding members/partners are reversibly masked to prevent interaction with their complementary binding moiety.

The mask may be provided by a detachable masking element. The masking element may be coupled to the binding agent via a selectively cleavable bond group or bond. The masking element may be located at or adjacent the binding member or binding partner. It may prevent binding to the cognate binding moiety via steric interference, electrostatic repulsion or any other suitable mechanism.

The binding moiety may be unmasked by selective cleavage of the masking element from the binding agent. A variety of techniques may be used, including, but not limited to irradiation (photolysis), oxidation, reduction, pH change, or enzymatic cleavage. The cleavage method may be selected appropriately depend upon the application for which the binding agent is to be used.

Use of enzymatic cleavage may enable a chain reaction of binding agent activation to take place. For example, a binding agent may comprise a catalytically inactive proenzyme (or zymogen) form of a protease, coupled to an antibody directed against the active protease but incapable of binding to the proenzyme. No aggregation of this binding agent will occur until the zymogen is activated by cleavage of its propeptide (e.g. by the presence of a catalytic amount of the active enzyme). The activated enzyme will then be capable of both activating the proenzyme of other binding agents and of binding to the antibody portion of the binding agent. Thus a chain reaction of activation and aggregation will be initiated. Suitable proenzymes include chymotrypsinogen.

In preferred embodiments the selectively cleavable group is cleavable by irradiation, e.g. by UV, infra-red, X-ray or visible irradiation. Laser irradiation may be particularly suitable, especially for therapeutic methods, as its delivery can be very closely controlled. Thus it can be used to irradiate only a site of diseased tissue (e.g. a tumour) without affecting surrounding healthy tissue.

Thus the binding member/partner is preferably masked to prevent interaction with its complementary binding moiety by a photocleavable moiety. Such photocleavable moieties are well known in the art. Protein binding agents can be suitably derivatised by means of appropriate reagents which couple to hydroxy or amino residues. Thus phosgene, diphosgene or DCCI (dicyclohexyl carbodiimide) may be used to generate photocleavable esters, amides, carbonates and the like from a wide variety of alcohols. Nitrophenyl derivatives may be used in this context. Substituted arylalkanols may be used, such as nitrophenyl methyl alcohol, 1-nitrophenylethan-1-ol, and substituted analogues. Thompson et al. (Biochem. Biophys. Res. Com. 201, 1213-1219 (1994) and Biochem. Soc. Trans. 225S, 23 (1995)) describe reversible inhibition of protein function by addition of 1-(2-nitrophenyl)-ethyl (NPE) moieties. Further photocleavable moieties will be well known to the skilled person, e.g. from “Biological Applications of Photochemical Switches”, H. Morrison (ed.), Bioorganic Photochemistry Series, Volume 2, J. Wiley & Sons. (see especially Chapter 1, section 4, pages 34 to 50). Other suitable photocleavable moieties include 1-(2-nitrophenyl)diazoethane (L. Bédouet et al., Recovery of the oxidative activity of caged bovine haemoglobin after UV photolysis, BBRC, 320 (2004) 939-944), 2-nitrophenylglycine (M. Endo et al, Design and synthesis of photochemically vontrollable caspase-3, Angew. Chem. Int. Ed 2004, 43, 5643-5645), 6-nitroveratryl (M. Endo et al, Design and synthesis of photochemically controllable restriction endonumclease BamHI by manipulation of the salt-bridge network in the dimmer interface, J. Org. Chem., 2004, 69, 4292-4289), o-nitrobenzyl and 4-hydroxyphenacyl.

Alternatively, the mask may be removed by a conformational change in the binding agent. This may create a new conformation in the binding agent which can be bound by the binding member, or may expose a part of the binding agent previously masked or screened (e.g. sterically). Thus the mask may reversed by a conformational change in the binding agent, whereby said binding member or said binding partner is exposed. Conformational change may take place along with, or as a result of, another method of activation. For example, in the proenzyme example described above, the antibody may recognise a particular conformation which is generated only on cleavage of the propeptide.

Complementary nucleic acid sequences may be used as binding moieties. A molecule containing two complementary sequences as binding moieties will self-hybridise so that it does not interact with other such molecules and so both moieties can be considered to be masked by this self-hybridisation. The molecule may be linear (in which case self-hybridisation will form a hairpin structure) or may be in the form of a closed loop. When the temperature of the system is raised above the melting temperature of the hairpin duplex, the hairpin will melt, providing free linear nucleic acid molecules in solution. As the temperature cools, sequences from different molecules will pair with one another, resulting in complex formation. The complementary sequences may have any desired sequence although complementary tracts of single nucleotides (e.g. poly(A) and poly(T), or poly(G) and poly(C)) may be particularly desirable. The complementary sequences may be separated by a linker sequence of any suitable length. Effector moieties may be conjugated to the molecule, particularly at the linker sequence. Such binding agents may be of DNA, RNA or any suitable nucleic acid analogue having a modified backbone, including protein nucleic acid (PNA). Typically, each complementary sequence will be at least 20 nucleotides in length, but any length may be suitable depending on the intended application.

### Effector Members

As described above, the nature and identity of the effector will depend upon the intended use of the binding agent.

Applications of the methods and binding agents described include signal generation and amplification. This may be particularly useful in assays for determining the presence or concentration of an analyte in a sample. Such assays typically employ agents having binding sites capable of specifically binding to the analyte of interest in preference to other molecules. Examples include antibodies, receptors and other molecules capable of specifically binding the analyte of interest. Conveniently, these agents are immobilised on solid supports, e.g. at defined, spatially separated locations, to make them easy to manipulate during the assay.

The sample is generally contacted with the binding agent(s) under appropriate conditions which allow the analyte in the sample to bind to the relevant agent(s).

The fractional occupancy of the binding sites of the binding agent(s) is then determined either by directly or indirectly labelling the analyte or by using a developing agent or agents to arrive at an indication of the presence or amount of the analyte in the sample. Typically, the developing agents are directly or indirectly labelled (e.g. with radioactive, fluorescent or enzyme labels, such as horseradish peroxidase) so that they can be detected using techniques well known in the art.

Directly labelled developing agents have a label associated with or coupled to the agent. Indirectly labelled developing agents may be capable of binding to a labelled species (e.g. a labelled antibody capable of binding to the developing agent) or may act on a further species to produce a detectable result.

Thus, radioactive labels can be detected using a scintillation counter or other radiation counting device, fluorescent labels using a laser and confocal microscope, and enzyme labels by the action of an enzyme label on a substrate, typically to produce a colour change. In further embodiments, the developing agent or analyte is tagged to allow its detection, e.g. linked to a nucleotide sequence which can be amplified in a PCR reaction to detect the analyte. Other labels are known to those skilled in the art are discussed below. The developing agent(s) can be used in a competitive method in which the developing agent competes with the analyte for occupied binding sites of the binding agent, or non-competitive method, in which the labelled developing agent binds analyte bound by the binding agent or to occupied binding sites. Both methods provide an indication of the number of the binding sites occupied by the analyte, and hence the concentration of the analyte in the sample, e.g. by comparison with standards obtained using samples containing known concentrations of the analyte.

There is also an increasing tendency in the diagnostic field towards miniaturisation of such assays, e.g. making use of binding agents (such as antibodies or nucleic acid sequences) immobilized in small, discrete locations (microspots) and/or as arrays on solid supports or on diagnostic chips. These approaches can be particularly valuable as they can provide great sensitivity (particularly through the use of fluorescent labelled reagents), require only very small amounts of biological sample from individuals being tested and allow a variety of separate assays can be carried out simultaneously. This latter advantage can be useful as it provides an assay employing a plurality of analytes to be carried out using a single sample. Examples of techniques enabling this miniaturised technology are provided in WO84/01031, WO88/1058, WO89/01157, WO93/8472, WO95/18376/WO95/18377, WO95/24649 and EP 0 373 203 A.

The methods, compositions and binding agents of the present invention can be used in numerous ways either to provide or to increase or amplify the signal obtained in such assays. These applications will be readily apparent to the skilled person.

Typically, for signal generation or amplification purposes (e.g. in assays and diagnostic applications), the effector comprises a signal generating means such as a label molecule, e.g. a radioactive, fluorescent, chemiluminescent or enzyme label, so that it can be detected using techniques well known in the art. Alternatively the binding agent may be indirectly labelled via a further labelled species (e.g. a labelled antibody capable of binding to the binding agent) in which case the effector may be viewed as that part of the binding agent with which the further labelled species interacts.

Different populations of binding agents in the same system may carry different label molecules. In preferred embodiments these different label molecules interact when their binding agents aggregate to give a different signal to that produced by the individual labels on the unaggregated binding molecules.

The signal generation methods described in The Immunoassay Handbook (Second Edition) Ed D Wild published by the Nature Publishing Group (2001) are particularly appropriate. Of particular application are those homogeneous systems described in Chapter 11 (E. F. Ullman). Scintillation Proximity Assay (SPA) (with for example a weak alpha or beta-emitter and a fluorophore) and Enzyme Channelling (with for example glucose oxidase and peroxidase) provide particularly attractive systems for use in the methods described. In these methods the binding agents may be labelled with complementary components of the detection system such that when they aggregate the two components are brought closely enough together to produce a detectable signal but when in solution prior to activation no such association occurs and thus no signal is produced.

Preferred enzyme labels are those capable of acting on a substrate to produce a detectable change. In preferred embodiments the change is detectable by spectrophotometric methods, e.g. a colour change. Alkaline phospahatase and horseradish peroxidase are well-known examples of such enzymes but the skilled person will be aware of other equally suitable examples.

The binding agents of the invention may also be used to generate or amplify a signal in a diagnostic method practised in vivo, e.g. to detect the presence of a tumour in a patient. For example, a tumour specific antibody may be administered to the patient in order to bind the tumour if present. An enzyme conjugated to the antibody may be capable of activating a labelled binding agent according to the invention in order to provide localisation of a detectable signal at the site of the tumour.

Other types of effector may be useful for therapeutic applications. The effector may play a part in localising the binding agent to a desired site and/or for activating particular biological effector functions. Binding agents may carry two or more of the same or different kinds of effector member.

Depending on the particular application, any biologically active molecule may be useful as an effector member. These include signalling molecules and other molecules capable of inducing a cellular response, such as a ligand for a cell surface receptor (e.g. a hormone, growth factor, cytokine etc.), molecules having binding functionalities, such as antibodies, lectins, receptors for ligands as described above, coreceptors, etc. and molecules having other biological effector functions such as enzymes, etc.

The present invention allows binding agents comprising such effectors to aggregate in response to a given stimulus, rapidly increasing the density of effector at the point of stimulus.

Molecules capable of binding to cell surface receptors may be used as effectors to induce particular responses from those cells. For example by cross-linking receptors on the surface of immune cells, those cells may be activated. Thus, particularly preferred are effectors capable of binding and optionally activating cells of the immune system. The examples below describe use of anti-CD3 antibodies to activate T cells, prompting them to proliferate and produce pro-inflammatory cytokines such as IL-2. When these antibodies are in individual form in solution they typically have little or no effect on T cells because of the limited scope for cross-linking of cell surface receptors which free individual antibodies provide. When used as part of binding agents of the present invention, though, these agents may be used to stimulate an immune response at a particular site in the body.

Further effector members include enzymes capable of activating prodrugs. Phosphatases, particularly alkaline phosphatase, can be used to convert phosphorylated prodrugs into a more cytotoxic de-phosphorylated form. For example, etoposide phosphate, mitomycin phosphate and doxorubicin phosphate may be dephosphorylated to yield etoposide, mitomycin and doxorubicin respectively. Carboxypeptidase, particularly G2, may be used to activate prodrugs in which a glutamic acid residue is used to inactivate the drug molecule. Beta-glucosidase can be used to produce cyanide from amygdalin. Beta-lactamases, such as penicillinase and cephalosporinase can be used to generate vinblastine or DAVLBHYD by hydrolysing the beta-lactam ring of a pro-drug comprising the drug linked to cephalosporin. Amidases, such as penicillin amidases, such as phenoxymethyl penicillin amidase can be used to produce melphalan or doxorubicin from their acetamide (e.g. phonoxyacetamide) derivatives. Cytosine deaminase can convert S-fluorocytisine to 5-fluorouracil. Nitroreductase, e.g. from E. coli, can be used to convert CB 1954 to an active alkylating agent.

The binding agent and prodrug may be administered to the subject separately or together, with activation of the drug taking place only at the desired site.

Alternatively the concentration of a drug at a particular site may be increased simply by using the drug molecule itself as the effector member, or by using an effector member having a particular affinity for the drug (e.g. an antibody against the drug).

An effector capable of binding to an antigen on the surface of a platelet may be used to promote platelet aggregation and thrombus formation at a site where clotting is desirable.

Effectors capable of binding to infectious agents may be used to cause aggregation of those agents, so reducing virulence, infectivity or pathogenicity, or increasing speed of clearance of the infectious agents by the immune system.

Effectors capable of binding to target cells may be used to localise one or more binding agents to that cell type. Such effectors may be used to localise binding agents carrying therapeutic effectors as described to the target cell type. Examples include binding agents (e.g. antibodies) directed against tumour specific antigens or parasite antigens such as viral proteins.

Binding agents according to the present invention may be used to form scaffolds for tissue engineering. This may be achieved by use of effectors capable of binding to one or more types of cell and/or extracellular matrix molecules.

The nature of the stimulus required to unmask the binding agent and trigger activation will control where and when aggregation occurs. For example, a photo-activatable binding agent may be induced to aggregate by local application of laser illumination. Alternatively a binding agent activatable by a metalloprotease may be activated in the locality of a metastatic tumour secreting that enzyme. This would not require any external stimulus to be applied after administration of the binding agent to the subject, but activation of the immune system would only occur at the disease site.

It will be appreciated that the effector member may also comprise one of the binding moieties via which aggregation takes place. Thus, when the effector is a label moiety such as fluorescein, the fluorescein may serve as one binding moiety while the other is an anti-fluorescein antibody. Where the effector is a signal-generating enzyme such as alkaline phosphatase, one of the binding moieties may be an anti-alkaline phosphatase antibody, while the other is the epitope on alkaline phosphatase to which the antibody binds. In general, it will be desirable that the effector is still able to carry out its effector function (e.g. fluorescence or catalytic activity) while bound by the other binding moiety.

### EXAMPLES

### Alkaline Phosphatase Signal Amplification Reagent

An antibody directed against alkaline phosphatase may be coupled to one or more alkaline phosphatase molecules with which it is immunologically reactive. Either the antigen binding site, the alkaline phosphatase, or both, may be masked e.g. by coating with a photocleavable moiety.

Such a reagent may be made by coating (masking) the antibody with e.g. NPE, as described by Thompson et al. (see above), and then conjugating it to alkaline phosphatase using conventional coupling techniques well known to those skilled in the art. This reagent may find application in immunoassays such as ELISAs in which a secondary antibody coupled to alkaline phosphatase (AP) is used. Instead of developing the assay by adding AP substrate after binding of the secondary antibody, the binding agent described above is added under conditions in which it is not activated. For example, where NPE is used as a coating reagent, simply keeping the reagent away from strong UV irradiation will keep it inactive. It is not generally necessary to use the reagent in the dark.

The binding agents are incapable of binding to either the immobilised AP or to each other until activated by irradiation. After irradiation it will aggregate on the immobilised AP. This will greatly increase the amount of AP immobilised at each bound secondary antibody, and will greatly increase the signal obtained when the assay is developed by addition of colour substrate. This achieves a number of benefits over conventional assays, including reduced development time and increased signal to noise ratio. Use of reagents of this sort is of particular benefit where small-scale assay systems are used, such as micro- or nano-scale array systems.

### Example 1

### Generation of NPE-Cloaked Alkaline Phosphatase—Anti-Alkaline Phosphatase Conjugate

A conjugate was prepared containing alkaline phosphatase conjugated to an NPE-coated anti-alkaline phosphatase antibody.

Alkaline Phosphatase (Biogenesis) was dialysed against 0.1M phosphate pH7.5 containing 0.1M NaCl. Its final concentration was 0.92 mg/ml.

Anti-Alkaline Phosphatase was obtained from Zymed. 2 mg of this antibody in 4 ml buffer was dialysed against 0.1 M Bicarbonate. Its final volume was 4.5 ml. 1.5 ml was retained as control.

NPE (1-(2-nitrophenyl)ethanol) (11 mg) was reacted with 7.8 μl diphosgene in 250 μl dry dioxan in the presence of 5.2 μl pyridine catalyst. A white precipitate immediately formed following which the mixture was left for 15 minutes before unreacted materials were evaporated away in a stream of nitrogen. The 1-(2-nitrophenol)ethoxycarbonyl chloride was resuspended in 250 μl dioxan for use.

10 μl of the carbonyl chloride (NPE-COCl) product were added to 2×1.5 ml aliquots of the antibody and left for 4 h. The solution was then dialysed overnight against 50 mM phosphate pH 7.5 and then centrifuged at 13K in a micro-centrifuge for 10 min. The final concentration of NPE-anti Alkaline Phosphatase was 0.26 mg/ml. OD 0.398 (giving an average of 7.2 residues of NPE per antibody molecule).

3-(2-pyridythio)propionic acid N-hydroxy succinimide ester (SPDP, Pierce), a bifunctional crosslinker, was used to conjugate the cloaked/coated antibody to the alkaline phosphatase.

1 ml of each of the Alkaline Phosphatase (0.5 mg/ml) and the NPE-coated anti-Alkaline Phosphatase antibody (0.26 mg/ml) in 0.05M phosphate buffer pH 7.5 were separately derivatised by the addition of a 60 fold molar excess of SPDP (60 μl and 30 μl respectively of 1.26 mg/ml SPDP in ethanol) for 2 h. Excess SPDP was then removed from each component on P10 desalting columns. The SPDP-Alkaline Phosphatase was then reduced by the addition of 100 μl 0.5M Dithiothreitol (DTT) for 30 minutes and the excess DTT was removed on another P10 column. The reduced SPDP-AP was then immediately added to the unreduced SPDP-derivatised NPE-anti-AP antibody and the mixture was left overnight at 20° C. to allow cross-linking to occur. The final protein concentration was 0.2 mg/ml.

### Use of NPE-Coated Anti-AP-AP Reagent in an Amplified Assay for Alkaline Phosphatase

Wells of a 96 well Elisa plate each received 100 μl of an alkaline phosphatase sample (comprising 0.01 μg/ml alkaline phosphatase in bicarbonate buffer pH9.6) and incubated overnight at 4° C. Following incubation the plate was blocked with 100 ul 0.5% BSA in coating buffer for 1 hour. The plate was then washed three times in PBS-Tween.

100 μl of the NPE-coated anti-AP-AP conjugate (0.01 mg/ml) was added to the wells, which were then UV irradiated for 0, 2, 5 and 10 minutes. After a further 1 h incubation the plate was washed and p-nitrophenyl phosphate substrate was added. Colour development was monitored at OD 405 nm. A greater than six-fold increase was obtained in the detection of the sample alkaline phosphatase after UV irradiation. The results are given in the table below.

| | | OD 405 nm minus |
|---|---|---|
| Irradiation time | OD 405 nm | background |
| (minutes) | (after 1 h) | (0.05) |
| 0 | 0.30 | 0.25 |
| 2 | 1.43 | 1.38 |
| 5 | 1.62 | 1.57 |
| 10 | 1.68 | 1.63 |

### Example 2

### Generation of NPE-Coated Biotin-Alkaline Phosphatase—Avidin Conjugates

A conjugate containing biotinylated alkaline phosphatase linked to NPE-coated avidin was prepared.

### Coating of Avidin with NPE

Avidin (from egg white, Sigma Chemical Co Ltd) at a concentration of 1 mg/ml was dialysed against 0.1M bicarbonate pH8.3 for 5 h during which the volume increased to 1.5 ml. 0.5 ml was retained as control.

NPE-COCl was prepared as described in example 1 above. 50 l NPE-COCl was added to the remaining 1 ml Avidin and left to gently rotate for 5 h. It was then dialysed against 10 mM phosphate pH 7.4 with 0.9% NaCl overnight followed by centrifugation at 13K for 10 min in a micro-centrifuge and the clear supernatant was taken. The resulting NPE-coated avidin had a protein conc. of 0.17 mg/ml and was coated with 28 residues of NPE per Avidin molecule.

### Biotinylation of Alkaline Phosphatase

10 mg of biotin and 10 mg of N-hydroxysuccinimide (NHS) are weighed out into the same tube and 500 ul of Dioxan is added. To this mixture is then added 8 mg of 1,3-dicyclohexylcarbodiimide (DCC) in 500 ul of Dimethylformamide (DMF). The solution is then left for 2.5 hours to react forming activated NHS-biotin esters. 100 ul of the NHS-biotin solution was then added to 2 ml of 1 mg/ml AP in 0.1M bicarbonate and left overnight to react. After centrifugation (13K for 10 min) and dialysis to remove uncoupled biotin, coating of the AP with biotin was confirmed by electrophoresis.

NPE-coated biotin-AP-avidin complexes were then generated by mixing 10 μg of the NPE-coated avidin with 50 μg of the AP-Biotin

### ELISA

The wells of a microtitre plate were coated with 100 μl of 0.0004 μg/ml Avidin in coating buffer pH9.6 overnight at 4° C. After overnight incubation the plate was blocked (0.5% BSA in coating buffer) for 1 hour then washed three times with PBS-Tween.

Equal volumes of 10 μg/ml Avidin-NPE and 50 μg/ml Biotin AP were mixed to give a complex of final concentration 5 ug/ml avidin-NPE/25 ug/ml of biotin-AP. 10 μl of this complex was then added to each well of the avidin coated ELISA plate. Half the plate was immediately irradiated with UV light for 3 minutes and the plate was incubated at 4° C. for 2 hours. After washing, p-nitrophenyl phosphate substrate was added to each well and colour development was monitored at 405 nm. An approx 5 fold increase in Avidin detection was obtained, with an OD 1.4 in irradiated wells compared to the unirradiated wells' OD of 0.32 after 20 minutes incubation. The proportional increase is even greater when the background value of OD 0.13 is subtracted from both values.

| Sample Avidin | | |
|---|---|---|
| Concentration | Non-illuminated | Illuminated |
| 0.4 pg/ml | 0.19 | 1.27 |

### Self-Aggregating T-Cell Activation Reagents

A murine anti-CD3 IgG and an anti-mouse IgG molecule may be separately coated, e.g. with NPE, before being conjugated to one another. The resulting conjugates are incapable of binding either to one another or to T cells. However after activation by irradiation, both the anti-IgG and anti-CD3 binding moieties are exposed. The resulting active conjugates will aggregate in solution by means of the IgG-anti-IgG interactions providing an extremely high local density of anti-CD3 sites capable of activating T cells.

This reagent may find application in the treatment of tumours, by stimulating T cell activation in their vicinity. The aggregate could be localised to the tumour via a further effector moiety capable of binding specifically to the tumour. Numerous variations are possible. For example the two antibodies could be masked with different photoactivatable moieties, allowing activation of the aggregation and T cell stimulating functions separately by different wavelengths of light. Alternatively, one of the antibodies could be masked by a different mechanism requiring a different stimulus. for example aggregation could be prevented by a peptide cleavable by an enzyme (e.g. a metalloenzyme) known to be secreted by the tumour. Aggregation would then occur on contact with the enzyme, and the T cell stimulating function would be separately activatable by targeted irradiation.

### Example 3

### T-Cell Activation

The murine anti-CD3 antibody OKT3 is coupled to Avidin by means of SPDP conjugation and the conjugate then coated with NPE as follows:

1-(2-nitrophenyl)ethoxycarbonylchloride (NPE-COCl) is prepared by dissolving 11 mg of NPE (1-(2-nitrophenyl)ethanol) NPE in 250 μl of dioxan followed by the addition of 5.6 μl pyridine as catalyst and then 7.8 μl diphosgene forming a white precipitate of the carbonylchloride. The solvent is then evaporated by nitrogen and the white solid resuspended in 250 μl fresh dioxan. 12 μl of this is then added to 0.5 mg of the OKT3-Avidin conjugate, wrapped in foil and left on a rotating stirrer for two hours. The product is dialysed overnight against PBS and centrifuged at 10,000 rpm for 10 minutes to remove aggregated material. The NPE-coated conjugate is then exposed to a 10-fold calculated excess of biotin over the avidin binding capacity had the avidin not been NPE-conjugated. The excess biotin is then removed by dialysis.

After this blocking step, the NPE-coated conjugate is biotinylated by conventional methods, e.g. as described above in Example 2.

The CD4-positive T-cell line H9, which expresses CD3, is obtained from ETCC (European Tissue Culture Centre). The cells are cultured in RPMI medium and 10% foetal calf serum until confluent providing a suspension of ca 10-6 cells/ml. The cells are then centrifuged at 1000 RPM for 5 minutes and re-suspended in medium (conditioned by previous OAW42 cell growth) at approximately half their original volume. 150 μl aliquots, containing ca 300,000 cells, are delivered to each well of a sterile microtitre plate. 30 μl of the conjugate to be tested, at a concentration of 0.1 mg/ml, is added to the H9 cells at this stage. A series is illuminated with UV light, and a series left un-illuminated.

Those wells to be irradiated by exposure to UV-A light are irradiated through the plastic cover of the plate with a VL-206BL UV-A lamp (2×6W tubes). The plate is then incubated for 3 hours at 37° C. The cells are then removed from the wells into Eppendorph tubes and centrifuged at 3000 RPM for 2 minutes. The cell supernatants are then decanted and IL-2 levels are then determined employing a sandwich ELISA kit (BD Biosciences, OptEIA Human IL-2 Set), for which an IL-2 standard curve is previously obtained over the range 15.6 pg/ml to 1000 pg/ml IL-2.

Comparative experiments and controls plus and minus illumination are also run as follows:

### (1) As above but with an added excess of free biotin (to inhibit cross-linking);

(2) no addition of NPE-conjugate;
(3) addition of native OKT3-avidin conjugate instead of biotinylated NPE-conjugate.

Good IL-2 production is found in those supernatants coming from cells exposed to the biotinylated NPE-conjugates illuminated by light.

### Example 4

### T-Cell Activation 2

As an alternative to Example 3, both a monoclonal rat anti-mouse IgG antibody and the murine anti-CD3 monoclonal antibody OKT3 are separately coated with NPE employing 1-(2-nitrophenyl)ethoxycarbonylchloride (NPE-COCl) as in example 3. Remaining free uncoated anti-mouse antibody is removed by specific absorption on a immunoaffinity column. The coated antibodies are then coupled to make a bispecific antibody and separated free from unconjugated antibodies. This light activatable bispecific antibody is then used in place of the NPE-OKT3-Avidin construct of Example 3 to demonstrate light activation of T-cell activation.

### Example 5

### Use of Light-Activatable Construct In Vivo

The light-activatable construct described in Example 4 can be used in in vivo T-cell activation as follows:

M5076 metastatic ovarian sarcoma is grown in BL6 mice. The tumour is subcutaneously transplanted into syngeneic animals as follows: the tumours are excised and diced as finely as possible in Hanks medium to provide a thick suspension 400 μl of which is mixed with 400 μl of the construct (at 50 μg/ml in Hanks medium) and then subcutaneously injected into the flank of the test animals. A separate group receives no added construct but only tumour.

Each group is then separated into two subgroups. One subgroup has no further manipulation, whereas the second has the skin around the subcutaneous injection irradiated with UV-A light (VL-206BL UV-A lamp (2×6W tubes) with a total UV-A irradiance of ca. 16 mW/cm2 at a distance of 1 cm.). After a number of weeks it will be seen that tumour growth is inhibited in the subgroup which have been given construct and been subjected to irradiation.

### Example 6

### Use of Zymogen Conjugate

A monoclonal antibody is raised against the cleavage site produced on chymotrypsin when it is cleaved from chymotrypsinogen. The monoclonal antibody should not react with chymotrypsinogen itself.

Mice are immunised with chymotrypsin and hybridomas generated by conventional methods. The resulting hybridomas are then screened for those producing antibodies against the chymotrypsin but not against chymotrypsinogen. This is done by coating the wells of microtitre plates with either chymoptrypsin or chymopstrypsinogen adding the candidate hybridoma supernatant culture fluid, allowing binding to occur, followed by the addition of a secondary labelled detector antibody. Those candidates giving rise to culture fluid which gives a significantly larger binding of secondary detector antibody are further cloned, screened and grown up to produce useful amounts of the antibody.

The antibody is conjugated to chymotrypsinogen to form a “secondary conjugate”, which can be used, e.g. in an immunoassay.

A “primary conjugate” is prepared by conjugating a detector antibody (directed against an antigen to be detected in the immunoassay) to both the antibody described above (reactive with chymotrypsin but not chymotrypsinogen) and to NPE-coated chymotrypsin. This is achieved by first making a bispecific antibody comprising both detector antibody and anti-chymotrypsin antibody, and then further conjugating NPE-inhibited chymotrypsin.

The immunoassay may be conducted in substantially conventional manner, e.g. in an ELISA plate having the analyte to be detected bound to it. After application and incubation of primary conjugate, unbound primary conjugate is washed from the plate and the secondary conjugate added.

The plate is then illuminated, activating the plate-bound chymotrypsin. The plate is incubated, during which time the chymotrypsin of the plate-bound primary conjugate acts on the chymotrypsinogen of the secondary conjugate to generate chymotrypsin. This chymotrypsin may then be bound by the anti-chymotrypsin of the primary conjugate as well as the anti-chymotrypsin of secondary conjugate molecules, and can in turn catalyse the conversion of chymotrypsinogen to chymotrypsin in other secondary conjugate molecules. This leads to a chain reaction of secondary conjugate activation and aggregation at those points on the surface of the plate at which primary conjugate is bound.

At a suitable time the reaction is stopped by removing the solution and washing the plate. The activity of chymotrypsin remaining bound to the plate is determined by addition of a suitable substrate (e.g. N-benzoyl-L-tyrosine ethyl ester). From standards provided on the plate, unknown samples can be determined.

### Example 7

An anti-Alkaline phosphatase antibody (Zymed) is cloaked with NPE as in Example 1 above.

A solution of Human Chorionic Gonadotrophin (HCG) of 1 ng/ml is prepared in 50 mM Tris buffer pH 7.4 and eleven five-fold serial dilutions made. Duplicate 50 μl aliquots of each of these, with a control blank of Tris buffer alone are then individually added into microtitre wells (the duplicates being well-spaced from each other) and incubated for 1 hr at room temperature in a humid chamber. The solutions are then shaken out and the wells washed four times with Tris Tween buffer.

50 μl of a 1:250 dilution of a 1 mg/ml anti-HCG antibody conjugated with alkaline phosphatase (Biogenis) is then added to each well, followed by 50 μl of a solution of 10 ng/ml NPE-conjugated anti-alkaline phosphatase antibody. Initial binding is allowed to take place by incubation for 30 minutes at room temperature in the dark. Then one of each set of duplicate wells is illuminated with a VL-206BL UV-A lamp (2×6W tubes), and the plate further incubated for 15 minutes at room temperature. Following this the solutions are shaken out of the plate and the plate washed four times with Tris Tween. p-nitrophenyl phosphate substrate is added to the wells and the optical density change is monitored at OD 405 nm. It is seen that with the well diluted HCG samples the optical density change in the illuminated well set is more rapid than the non illuminated allowing faster and more sensitive detection.

### Example 8

### Example 8

A human anti-mouse IgG is cloaked with NPE employing 1-(2-nitrophenyl)ethoxycarbonylchloride (NPE-COCl) as in example 3 above, such that the antibody does not bind mouse IgG until illuminated.

The CD4-positive T-cell line H9 is cultured in RPMI medium and 10% foetal calf serum until confluent. The cells are then centrifuged at 1000 RPM for 5 minutes and re-suspended in medium (conditioned by previous OAW42 cell growth) at approximately half their original volume. 150 μl aliquots, containing ca 300,000 cells, are delivered to each well of a sterile microtitre plate to provide two series of wells. Both receive either 20 μl of an equal mixture of OKT3 antibody and NPE-treated human anti-mouse antibody both at 0.1 mg/ml or 20 μl of 0.05 mg/ml OKT3 antibody.

One series is illuminated by exposure to UV-A light by irradiation through the plastic cover of the plate with a VL-206BL UV-A lamp (2×6W tubes) and the other series left un-illuminated.

The plate is then incubated for 3 hours at 37° C. The cells are then removed from the wells into Eppendorph tubes and centrifuged at 3000 RPM for 2 minutes. The cell supernatant solutions are then decanted and IL-2 levels are then determined employing a sandwich ELISA kit (BD Biosciences, OptEIA Human IL-2 Set), for which an IL-2 standard curve is previously obtained over the range 15.6 pg/ml to 1000 pg/ml IL-2.

Those wells receiving both the NPE-treated human anti-mouse antibody and OKT3 plus illumination are found to produce more IL2 than the other wells.

While the invention has been described in conjunction with the exemplary embodiments described above, many equivalent modifications and variations will be apparent to those skilled in the art when given this disclosure. Accordingly, the exemplary embodiments of the invention set forth are considered to be illustrative and not limiting. Various changes to the described embodiments may be made without departing from the spirit and scope of the invention. All references cited herein are expressly incorporated by reference.

1. A binding agent comprising two binding moieties, the binding moieties comprising
(i) a binding member, capable of binding to a binding partner, and
(ii) the binding partner,
one of said binding moieties being reversibly masked, whereby two binding agents comprising said binding moieties do not bind one another.

2. A binding agent according to claim 1 wherein the binding member and binding partner of a single binding agent cannot interact with one another after the reversibly masked binding moiety is unmasked.

3. A binding agent according to claim 2 wherein the binding partner and binding member are sterically incapable of interacting with one another.

4. A binding agent according to claim 1 wherein the binding moiety is reversibly masked by a detachable masking element.

5. A binding agent according to claim 4 wherein the masking element is coupled to the binding agent via a selectively cleavable group or bond.

6. A binding agent according to claim 5 wherein the selectively cleavable group or bond is cleavable by irradiation, oxidation, reduction, pH change, or enzymatic cleavage.

7. A binding agent according to claim 6 wherein the irradiation is UV irradiation.

8. A binding agent according to claim 5 wherein the selectively cleavable group or bond is cleavable by a protease.

9. A binding agent according to claim 8 wherein cleavage of the selectively cleavable group or bond activates a protease activity of the binding agent.

10. A binding agent according to claim 2 wherein said binding moiety may be unmasked by a conformational change in the binding agent.

11. A binding agent according to claim 1 wherein the binding moieties are an antibody and its cognate epitope.

12. A binding agent according to claim 1 wherein the binding moieties are avidin or streptavidin and biotin.

13. A binding agent according to claim 1 further comprising an effector member.

14. A binding agent according to claim 13 wherein the effector member is a signal generating means.

15. A binding agent according to claim 14 wherein the signal generating means is a label moiety or an enzyme.

16. A binding agent according to claim 13 wherein the effector member has a binding functionality.

17. A binding agent according to claim 16 wherein the effector member is capable of binding to a target cell type.

18. A binding agent according to claim 17 wherein the effector member is capable of binding to a tumour specific antigen or a parasite antigen.

19. A binding agent according to claim 17 wherein the target cell is a cell of the immune system.

20. A binding agent according to claim 19 wherein the effector member is capable of activating said cell of the immune system.

21. A binding agent according to claim 20 wherein the cell is a T cell.

22. A binding agent according to claim 22 wherein the effector member is an anti-CD3 antibody.

23. A binding agent according to claim 13 wherein the effector member is an enzyme.

24. A binding agent according to claim 23 wherein the enzyme is capable of converting a prodrug to an active form.

25. A binding agent according to claim 13 wherein the effector member is reversibly masked.

26. A binding agent according to claim 13 wherein the effector member is unmasked by the same means as the binding member or partner.

27. A binding agent according to claim 13 comprising at least two different effector members.

28-29. (canceled)

30. A method of making a binding agent comprising the steps of:
(i) providing a first component comprising a binding member, capable of binding to a binding partner; and
(ii) providing a second component comprising said binding partner,
wherein one of said binding member and binding partner is reversibly masked such that said binding member or partner is prevented from binding to the other; and
(iii) contacting said first component with said second component such that they become associated with one another.

31. A method according to claim 30 wherein the first and second components are covalently linked to one another.

32. A method according to claim 30 wherein the first and second components are non-covalently associated with one another.

33. A method according to claim 32 wherein the first and second components respectively comprise avidin/streptavidin and biotin.

34. A composition comprising at least two populations of binding agents, each population having at least two binding members for respective binding partners, each of said binding partners being present on another of said populations of binding agents, wherein a binding member of at least one of said populations is reversbily masked, whereby said one population does not bind the population carrying the respective binding partner.

35. A method of causing aggregation of a plurality of binding agents, comprising:
providing a composition comprising at least two populations of binding agents, each population having at least two binding members for respective binding partners, each of said binding partners being present on another of said populations of binding agents, wherein a binding member of at least one of said populations is reversibly masked, whereby said one population does not bind the population carrying the respective binding partner,
and masking said binding member, whereby said one population becomes capable of binding the population carrying the respective binding partner.

36. A kit comprising at least two populations of binding agents, each population having at least two binding members for respective binding partners, each of said binding partners being present on another of said populations of binding agents, wherein a binding member of at least one of said populations is reversibly masked, whereby said one population does not bind the population carrying the respective binding partner.

37. A method of determining an analyte in a sample, the method comprising:
(a) contacting the sample with a plurality of binding agents each comprising two binding moieties, the binding moieties comprising:
(i) a binding member, capable of binding to a binding partner, and
(ii) the binding partner,
one of said moieties being reversibly masked, whereby said binding agents do not bind one another;
(b) contacting the sample with a detecting agent capable of binding to the analyte if present, wherein the detecting agent comprises one of said binding moieties; and
(c) unmasking said reversibly masked binding moiety, whereby said binding agents form an aggregate.

38. A method according to claim 37 wherein the detecting agent is one of the binding agents.

39. A method according to claim 37 comprising the step of detecting the presence of aggregated binding agents.

40. A method according to claim 39 wherein the binding agents comprise signal generating means.

41. A method according to claim 37 wherein the analyte is immobilised on a solid phase.

42. A method according to claim 37 wherein the analyte is a protein, polypeptide, peptide, carbohydrate, nucleic acid, organic or inorganic polymer, or a small organic or inorganic molecule.

43. A method according to claim 37 wherein the detecting agent is an antibody specific for the analyte.

44. A method according to claim 43 which is an ELISA, Western blot, immunohistochemistry, or immunofluorescence assay.

45. A method according to claim 37 wherein the detecting agent is a nucleic acid molecule capable of hybridising to the analyte.

46. A method according to claim 45 which is a Southern blot, Northern blot or in situ hybridisation.

47. A method of determining an analyte in a sample, the method comprising
(a) contacting the sample with
(i) a competitor, and
(ii) a plurality of binding sites, wherein each binding site is capable of binding to the analyte if present and to the competitor, but not to both simultaneously;
(b) contacting the sample with a plurality of binding agents each comprising two binding moieties, the binding moieties comprising
(i) a binding member, capable of binding to a binding partner, and
(ii) the binding partner,
one of said binding moieties being reversibly masked, whereby said binding agents do not bind one another; and
(c) contacting the sample with a detecting agent capable of binding to the competitor if present, wherein the detecting agent comprises one of said binding moieties; and
(d) unmasking said reversibly masked binding moiety, whereby said binding agents form an aggregate.

48. A method of determining an analyte in a sample, the method comprising
(a) contacting the ample with at least two populations of binding agents, each population having at least two binding members for respective binding partners, each of said binding partners being present on another of said populations of binding agents, wherein a binding member of at least one of said populations is reversibly masked, whereby said one population does not bind the population carrying the respective binding partner,
(b) contacting the sample with a detecting agent capable of binding to the analyte if present, wherein the detecting agent comprises one of said binding members or binding partners, and
(c) unmasking said reversibly masked binding member, wherein said binding agents form an aggregate.

49. A method of determining an analyte in a sample, the method comprising
(a) contacting the sample with
(i) a competitor, and
(ii) a plurality of binding sites, wherein each binding site is capable of binding to the analyte if present and to the competitor, but not to both simultaneously;
(b) contacting the sample with at least two populations of binding agents, each population having at least two binding members for respective binding partners, each of said binding partners being present on another of said populations of binding agents, wherein a binding member of at least one of said populations if reversibly masked, whereby said one population does not bind the population carrying the respective binding partner;
(c) contacting the sample with a detecting agent capable of binding to the competitor if present, wherein the detecting agent comprises one of said binding members or binding partners; and
(d) unmasking said reversibly masked binding moiety, whereby said binding agents form an aggregate.

50. A method of causing aggregation of a therapeutic agent at a physiological site, the method comprising administering a plurality of binding agents to an individual, each of said binding agents comprising two binding moieties, the binding moieties comprising
(i) a binding member, capable of binding to a binding partner, and
(ii) the binding partner;
one of said binding moieties being reversibly masked, whereby said binding agents do not bind one another,
each of said binding agents further comprising said therapeutic agent,
and wherein the method further comprises the step of inducing aggregation of said binding agents by unmasking said reversibly masked binding moiety such that said binding agents bind one another.

51. A method according to claim 50 wherein the physiological site is a disease site.

52. A method according to claim 51 wherein the disease site is a tumour or a site of parasite infection.

53. A method according to claim 50 wherein the physiological site is the site of a wound.

54. A method according to claim 50 comprising administering a targeting agent capable of binding to a molecule which is expressed at the site and capable of binding to one of the binding agents or capable of unmasking said binding moiety.

55. A method according to claim 50 wherein the binding moiety is unmasked by illumination or enzyme action.

56. A method according to claim 50 wherein the therapeutic agent is capable of binding to a molecule expressed on a cell surface, a molecule capable of activating a cell of the immune system, a drug or prodrug molecule, or an enzyme.

57. A method according to claim 56 wherein the therapeutic agent is an anti-CD41 antibody, and anti-CD3 antibody or an enzyme capable of converting a prodrug to an active form.

58. A method according to claim 50 wherein the therapeutic agent is reversibly unmasked and can be unmasked by the same mechanism as the binding moiety.

59. A method of causing aggregation of a therapeutic agent in a culture of cells, the method comprising contacting a culture of cells with a plurality of binding agents, each of said binding agents comprising two binding moieties, the binding moieties comprising
(i) a binding member, capable of binding to a binding partner, and
(ii) the binding partner,
one of said binding moieties being reversibly masked, whereby said binding agents do not bind one another,
each of said binding agents further comprising said therapeutic agent,
and wherein the method further comprises the step of inducing aggregation of said binding agents by unmasking said reversibly masked binding moiety such that said binding agents bind one another.

60. A method according to claim 59 wherein the cells comprise cancer cells, parasitically-infected cells, cells of the immune system or platelets.
